(19) United States
(12) Patent Application Publication
(10) Pub. No.: US 2008/0260849 A1
(43) Pub. Date: Oct. 23, 2008

(54) COMPOSITION FOR TRANSMUCOSAL ABSORPTION

(75) Inventors: Makiko Aimi, Kanagawa (JP); Kazutaka Ogiwara, Kanagawa (JP); Shouji Ooya, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/105,409

(22) Filed: Apr. 18, 2008

(30) Foreign Application Priority Data
Apr. 19, 2007 (JP) 2007-110037

(51) Int. Cl. A61K 9/14

(52) U.S. Cl. 424/499

(57) ABSTRACT

It is an object of the present invention to provide a composition for transmucosal absorption which comprises highly safe protein nanoparticles having high transparency due to the small particle size and high transmucosal absorbability. The present invention provides a composition for transmucosal absorption which comprises protein nanoparticles containing an active ingredient and having an average particle size of 10 nm to 300 nm.

### TECHNICAL FIELD

The present invention relates to a composition for transmucosal absorption which comprises protein nanoparticles containing an active ingredient.

### BACKGROUND ART

The drugs for pharmaceutical product which have been widely used include many poorly absorbable drugs which show very low transmucosal absorbability such as peptide hormones and β-lactam antibiotics. Typical examples of such drugs include insulin (which is peptide hormones) and ampicillin (which is β-lactam antibiotics).

Insulin is an effective drug as a therapeutic agent for diabetes. However, due to the aforementioned reasons, insulin has been administered to a patient via injection. However, injection administration of insulin gives pain to a patient at the time of administration. Also, long-term continuous administration causes some problems such as thickening of tissue at injection site.

In order to avoid these problems and realize an administration at home, administration route of drugs has been extensively studied, and preparations which utilize skin or mucosa as administration route such as transdermal and transpulmonary administration have been studied. These administration methods have advantages that bioavailability is high; compliance of patients is high; stop of administration at excessive administration is easy; and administration to a physically-challenged patient is easy. In view of these advantages, study has been made to formulate an insulin preparation as suppository or nasal preparation by using insulin which has large molecular weight and is poorly absorbable in combination with a substance which can promote insulin absorption via mucosa such as rectal mucosa and nasal mucosa. However, both of promoting effect of transmucosal absorption and safety were not sufficient.

Japanese Patent Publication (Kokai) 2006-28031 proposes nanoparticles comprising biodegradable polymer such as lactic acid/glycolic acid copolymer (PLGA) and lactic acid polymer (PLA), which incorporates a drug for transmucosal absorption. However, these polymers are likely to be hydrolyzed, and the preservation stability is low. Further, when the polymer is decomposed in living body, lactic acid is generated, side effect is concerned.

### DISCLOSURE OF THE INVENTION

It is an object of the present invention to solve the above problems of the prior art. Specifically, it is an object of the present invention to provide a composition for transmucosal absorption which comprises highly safe protein nanoparticles having high transparency due to the small particle size and high transmucosal absorbability.

As a result of intensive studies in order to achieve the above object, the present inventors demonstrated that protein nanoparticles containing an active ingredient, which were prepared by the present inventors, are highly safe and have high transparency and favorable permeability into mucosa. The present invention has been completed based on the above findings.

Thus, the present invention provides a composition for transmucosal absorption which comprises protein nanoparticles containing an active ingredient and having an average particle size of 10 nm to 300 nm.

Preferably, the composition for transmucosal absorption of the present invention contains protein nanoparticles in an amount of 1% to 50% by weight.

Preferably, the protein nanoparticles contain an active ingredient in a weight that is 0.1% to 100% of the protein weight.

Preferably, the active ingredient is at least one selected from the group consisting of functional food ingredients, and pharmaceutical ingredients.

Preferably, the active ingredient is an ionic substance or a fat-soluble substance.

Preferably, the protein is at least one selected from the group consisting of collagen, gelatin, acid-treated gelatin, albumin, ovalbumin, casein, transferrin, globulin, fibroin, fibrin, laminin, fibronectin, and vitronectin.

Preferably, the protein is subjected to crosslinking treatment during and/or after nanoparticle formation.

Preferably, crosslinking treatment is carried out by an enzyme.

The enzyme is not particularly limited so long as it has a function of crosslinking a protein, and preferably transglutaminase is used.

Preferably, the composition for transmucosal absorption according to the present invention comprises casein nanoparticles prepared by the following steps (a) to (c):

(a) mixing casein with a basic aqueous medium at pH of from 8 to less than 11;
(b) adding at least one active ingredient to the solution obtained in step (a); and
(c) injecting the solution obtained in step (b) into an aqueous medium at pH of 3.5 to 7.5:

Preferably, the composition for transmucosal absorption according to the present invention comprises casein nanoparticles prepared by the following steps (a) to (c):

(a) mixing casein with a basic aqueous medium at pH of from 8 to less than 11;
(b) adding at least one active ingredient to the solution obtained in step (a); and
(c) lowering the pH of the solution obtained in step (b) to pH value which is different from the isoelectric point by 1 or more units.

Particles containing an active ingredient in the composition for transmucosal absorption according to the present invention are nanoparticles, and thus they are highly absorbable. In addition, according to the present invention, since protein nanoparticles are used, there is no need to use chemical crosslinking agents or synthetic surfactants for its production, and therefore the composition of the present invention is highly safe.

### BRIEF DESCRIPTION OF THE DRAWINGS

FIG. 1 shows the results of heat stability test of Test Example 2.

### BEST MODE FOR CARRYING OUT THE INVENTION

Hereafter, embodiments of the present invention will be specifically described.

The composition for transmucosal absorption of the present invention comprises protein nanoparticles containing an active ingredient and having an average particle size of 10 nm to 300 nm.

The active ingredients used in the present invention is not particularly limited, so long as it is absorbed via mucosa and shows its activity. The active ingredients can be selected from, for example, functional food ingredients or pharmaceutical ingredients. Examples of the functional food ingredients may include minerals, antioxidants, anti-stress agents, vitamin agents, radical oxygen scavengers, nutritious supplements, amino acids, carotenoid, fruit and plant extracts, skin-lightening agents, hair growth agents, hair nutritional supplements, hair growth stimulants, anti-gray hair agents, anti-aging agents, collagen synthesis promoters, anti-wrinkle agents, anti-acne agents, melanin generation suppressing agents, melanocyte activating agents, and slimming agents. Examples of the pharmaceutical ingredients may include steroids, antibiotics, anti-cancer agents, anti-inflammatory agents, antiallergenic agents, antimicrobial agents, hormone agents, antithrombotic agents, immune-suppressing agents, therapeutic agents for skin diseases, antimycotic agents, nucleic acid drugs, anesthetic agents, antipyretic agents, analgesic agents, antipruritic agents, anti-edema agents, antitussive and expectorant agents, antiepileptic agents, antiparkinson agents, hypnotic sedative agents, antianxiety agents, analeptic agents, psychoneurotic agents, muscle relaxants, antidepressants, hair growth agents, hair nutritional supplements, hair growth stimulants, combination cold remedies, automatic nerve agents, spasmolytic agents, diaphoretic agents, antiperspirant agents, cardiotonic agents, antiarrhythmic agents, vasoconstrictive agents, vasodilating agents, antihypertensive agents, therapeutic agents for diabetes, hyperlipidemia agent, respiratory stimulants, cough medicines, β blocker, α blocker, α β blocker, miotic agents, mydriatic agents, prostaglandin, vitamins, agents for parasitic skin diseases, homeostasis agents, vaccines, physiologically active peptides and proteins, antibodies, and antigens. The active ingredients as mentioned above may be used alone or in combination.

Examples of antioxidants used in the present invention may include carotenes, retinoic acid, retinol, vitamin C and derivatives thereof, kinetin, astaxanthin, tretinoin, vitamin E and derivatives thereof, sesamin, α-lipoic acid, coenzyme Q10, flavonoids, erythorbic acid, gallic acid propyl, BHT (di-n-butylhydroxytoluene), BHA (butylhydroxyanisole), Engelhardtia chrysolepis Hance extract, soybean extract, black tea extract, green tea extract, and Rosae multiflorae fructus extract, but are not limited thereto.

Examples of vitamins used in the present invention may include vitamin A and a derivative thereof, retinoic acid, vitamin B family (e.g., vitamin B1, vitamin B2, vitamin B6, vitamin B12, and folic acid), vitamin C and a derivative thereof, vitamin D, vitamin E, vitamin F, pantothenic acid, and vitamin H, but are not limited thereto.

Examples of radical oxygen scavengers used in the present invention may include superoxide dismutase (SOD), mannitol, carotenoids such as beta carotene, astaxanthin, rutin and derivatives thereof, bilirubin, cholesterol, tryptophan, histidine, quercetin, quercitrin, catechin, catechin derivatives, gallic acid, gallic acid derivatives, Scutellariae radix extract, ginkgo extract, Saxifraga stolonifera (strawberry geranium) extract, melissa extract, Geranium thunbergii extract, moutan cortex extract, parsley extract, tormentilla extract, Momordica grosvenori extract, seaweed extract, “Yashajitsu” (Alnus firma Sieb. et Zucc.) extract, and Lycii cortex extract, but are not limited thereto.

Examples of hair growth agents used in the present invention may include finasteride, minoxidil or an analog thereof, adenosine, cepharanthin, glycyrrhetic acid or a derivative thereof, glycyrrhizin acid or a derivative thereof, isopropyl methyl phenol, pantothenic acid, panthenol, t-flavanone, tocopherols or a derivative thereof, hinokitiol, pentadecanoic acid or a derivative thereof, licorice extract, Lepisorus extract, sophora root extract, swertia herb extract, capsicum extract, Ampelopsis cantoniensis var. grossedentata extract, carrot extract, Taraxacum extract, tree peony extract, and orange extract, but are not limited to thereto.

Examples of antiaging agents used in the present invention may include retinoic acid, retinol, vitamin C and a derivative thereof, kinetin, β-carotene, astaxanthin, and tretinoin, but are not limited thereto.

Examples of antibiotics used in the present invention may include penicillin-based antibiotics (for example, penicillin G, penicillin V, methicillin, oxacillin, cloxacillin, ampicillin, hetacillin, ciclacillin, amoxicillin, carbenicillin and sulbenicillin), cephalosporin-based antibiotics (for example, cephaloridine, cephalothin, cefazolin, cephaloglycin, and cefalexin), aminoglucoside based antibiotics (for example, streptomycin, Kanamycin, dibekacin, gentamicin, and fradiomycin), tetracycline-based antibiotics (for example, oxytetracycline, tetracycline, dimethylchloro tetracycline, doxycycline, and minocycline), macrolide-based antibiotics (for example, erythromycin, leucomycin, and Josamycin), lincomycin-based antibiotics (for example, lincomycin and clindamycin), chloramphenicol, mikamycin, gramicidin, gramicidin S, capreomycin, cycloserine, enviomycin, rifampicin, nystatin, trichomycin, amphotericin B, griseofulvin, variotin, PyrroInitrin, siccanin, nitrofurantoin, 5-iodo-2-deoxyuridine, cefamezin, phosphonomycin, and N-formimideylthenamycin monohydrate, but are not limited thereto.

Examples of anticancer agents used in the present invention may include fluorinated pyrimidine antimetabolites (for example, 5-fluorouracil (5-FU), tegafur, doxifluridine, and capecitabine); antibiotics (for example, mitomycin (MMC) and adriacin (DXR)); purine antimetabolites (for example, folic acid antagonists such as methotrexate and mercaptopurine); active metabolites of vitamin A (for example, antimetabolites such as hydroxy carbamide, tretinoin, and tamibarotene); molecular targeting agents (for example, Herceptin and imatinib mesylate); platinum agents (for example, Briplatin or Randa (CDDP), Paraplatin (CBDC), Elplat (Oxa), and Akupura); plant alkaloids (for example, Topotecin or Campto (CPT), taxol (PTX), Taxotere (DTX), and Etoposide); alkylating agents (for example, busulphan, cyclophosphamide, and ifomide); antiandrogenic agents (for example, bicalutamide and flutamide); estrogenic agents (for example, fosfestrol, chlormadinone acetate, and estramustine phosphate); LH-RH agents (for example, Leuplin and Zoladex); antiestrogenic agents (for example, tamoxifen citrate and toremifene citrate); aromatase inhibitors (for example, fadrozole hydrochloride, anastrozole, and exemestane); progestational agents (for example, medroxyprogesterone acetate); and BCG, but are not limited thereto.

Examples of an antiinflammatory agent used in the present invention may be non-steroid antiinflammatory agents or steroid antiinflammatory agents, and may include a compound which is selected from hydrocortisone, prednisolone, fluocinolone acetonide, fluoroxycortide, methylprednisolone, hydrocortisone acetate, triamcinolone acetonide, dexamethasone, betamethasone acetate, diflucortolone valerate, clobetasol propionate, fluocinonide, azulene, guaiazulene, diphenhydramine hydrochloride, glycyrrhizinic acid, glycyrrhetinic acid, mefenamic acid, phenylbutazone, indometacin, ibuprofen and ketoprofen, and its derivative and its salt; and a plant extract which is selected from Scutellariae Radix extract, Artemisia capillaris Thunb. Extract, Platycodon grandiflorum extract, Armeniacae Semen extract, Common gardenia extract, Sasa veitchii extract, Gentiana lutea extract, Comfrey extract, white birch extract, Malva extract, Persicae Semen extract, peach blade extract, and loquat blade extract, but are not limited thereto.

Examples of antiallergic agents used in the present invention may include mediator antireleasers, such as disodium cromoglycate and tranilast; histamine H₁ antagonists, such as ketotifen fumarate and azelastine hydrochloride; thromboxane inhibitors, such as ozagrel hydrochloride; leukotriene antagonists, such as pranlukast; and suplatast tosylate, but are not limited thereto.

Examples of antimicrobial agents used in the present invention may include ofloxacin, levofloxacin, norfloxacin, lomefloxacin hydrochloride, sulbenicillin sodium, gentamicin sulfate, micronomicin sulfate, piroctone olamine, isopropyl methyl ether, hinokitioru, zinc pyrithione, climbazole, benzalkonium chloride, photosensitive dye 101, photosensitive dye 201, chlorhexidine, salicylic acid, phenol, ketoconazole and miconazole, but are not limited thereto.

Examples of hormone agents used in the present invention may include estradiol, ethynyl estradiol, estrin, cortisone, hydrocortisone, prednisone and prednisolone, but are not limited thereto.

Examples of antithrombotic agents used in the present invention may include aspirin, ticlopidine hydrochloride, cilostazol, and warfarin potassium, but are not limited thereto.

Examples of immunosuppressive agents used in the present invention may include rapamycin, tacrolimus, ciclosporin, prednisolone, methylprednisolone, mycophenolate mofetil, azathioprine, and mizoribine, but are not limited thereto.

The antimycotic agents used in the present invention are a substance which inhibits growth of fungus or kills fungus, and example thereof may include undecylenic acid, zinc undecylenate, salicylic acid, antitol, mokutal, siccanin, trichomycin, nystatin, pyrroInitrin, variotin, sulfur, cloconazole hydrochloride, clotrimazole, iconazole nitrate, econazole nitrate, oxyconazole nitrate, sulconazole nitrate, miconazole nitrate, tioconazole, exalamide, biphonazole, and phenyliodo undecynoate, but are not limited thereto.

Examples of nucleic acid drugs used in the present invention may include antisense, ribozyme, siRNA, aptamer, and decoy nucleic acid, but are not limited thereto.

Examples of anesthetic agents used in the present invention may include benzocaine, procaine, lidocaine, and tetracaine, but are not limited thereto.

Examples of antipyretic agents used in the present invention may include any known compound having antipyretic action, but are not limited thereto.

Examples of analgesic agents used in the present invention may include any known compound having analgesic action, but are not limited thereto.

Examples of antitussive and expectorant agents used in the present invention may include procaterol hydrochloride, terbutaline sulfate, fenoterol hydrobromide, tulobuterol hydrochloride, ambroxol hydrochloride, pirbuterol hydrochloride, mabuterol hydrochloride, clenbuterol hydrochloride, trimetoquinol hydrochloride, formoterol fumarate, but are not limited thereto.

Examples of vasodilating agents used in the present invention may include efloxate, etafenone, oxyfedrine, carbochromen, dilazep, diltiazem, trimetazidine, pentaerythritol tetranitrate, dipyridamole, isosorbide nitrate, trapidil, nitroglycerin, nifedipine, prenylamine, molsidomine, troInitrate phosphate, inositol hexanicotinate, isoxsuprine, nylidrin, nicametate citrate, cyclandelate, cinnarizine, nicotinic alcohol, and hepronicato, but are not limited thereto.

Examples of antihypertensive agents used in the present invention may include rauwolfia alkaloids (for example, reserpine and rescinnamine), clonidine, prazosin, dihydroergotoxine mesylate, meticrane, methyldopa, guanethidine and betanidine, but are not limited thereto.

Examples of the physiologically active peptides and proteins, antibodies, vaccines and antigens, which are used in the present invention may include calcitonin, insulin, proinsulin, vasopressin, desmopressin, luteinizing hormone, luteinizing hormone-releasing hormone, somatostatin, prolactin, glucagons, gastrin, secretin, kallikrein, urokinase, neurotensin, enkephalin, Kyotorphin, endorphin, endothelin, angiotensin, transferring, atrial natriuretic peptide, epidermal growth factor, growth hormone, parathyroid hormone, interferon, interleukin, tumor necrosis factor, leukemia cell inhibitor, blood stem cell growth factor, erythropoietin, granulocyte colony-stimulating factor, granulocyte macrophage-stimulating factor, macrophage colony stimulating factor, thrombopoietin, super oxide dismtase, tissue plasminogen activator, antithrombin, blood coagulation factor, anti-IgE antibody, anti-IgA antibody, anti-tumor antibody, anti-tumor necrosis factor antibody, anti-interleukin antibody, HIV-neutralizing antibody, anti-platelet antibody, anti-hepatitis virus antibody, hepatitis vaccine, influenza vaccine (influenza antigen), pertussis vaccine, diphtheria vaccine, tetanus vaccine, peptide and proteins capable of acting as antigen such as Japanese cedar pollen and hogweed pollen, and hapten bound product thereof, and a mixture of it with adjuvant, but are not limited thereto.

According to the present invention, it was found that, with the use of interaction between a fat-soluble active ingredient and a casein hydrophobic domain, it is possible for casein nanoparticles to contain the active ingredient. Further, it was found that such particles remain stable in an aqueous solution.

Further, it was found that a particle mixture of casein and ionic polysaccharide or another ionic protein can contain an ionic active ingredient.

The composition for transmucosal absorption of the present invention comprises preferably 0.01% to 50% by weight and most preferably 0.1% to 10% by weight protein nanoparticles.

The composition for transmucosal absorption of the present invention contains an active ingredient in a weight that is preferably 0.1% to 100% and more preferably 0.1% to 50% of the protein weight.

According to the present invention, an active ingredient may be added during or after protein nanoparticle formation.

The average particle size of protein nanoparticles used in the present invention is generally 10 to 300 nm, preferably 10 to 200 nm, more preferably 10 to 100 nm, and particularly preferably 20 to 50 nm.

The type of protein used in the present invention is not particularly limited. However, a protein having a lysine residue and a glutamine residue is preferable. In addition, such protein having a molecular weight of approximately 10,000 to 1,000,000 is preferably used. The origin of the protein is not particularly limited. However, a human-derived protein is preferably used. Specific examples of a protein that can be used may include at least one selected from the group consisting of collagen, gelatin, acid-treated gelatin, albumin, ovalbumin, casein, transferrin, globulin, fibroin, fibrin, laminin, fibronectin, and vitronectin. However, the compound used in the present invention is not limited to the aforementioned compounds. In addition, the origin of the protein is not particularly limited. Thus, bovine, swine, fish or plant protein, as well as recombinant protein of any thereof, can be used. Examples of recombinant gelatin that can be used include, but are not limited to, gelatins described in EP1014176 A2 and U.S. Pat. No. 6,992,172. Among them, casein, acid-treated gelatin, collagen, or albumin is preferable. Further, casein or acid-treated gelatin is most preferable. When casein is used in the present invention, the origin of the casein is not particularly limited. Casein may be milk-derived or bean-derived. Any of α-casein, β-casein, γ-casein, and κ-casein, as well as a mixture thereof, can be used. Caseins may be used alone or in combinations of two or more.

Proteins used in the present invention may be used alone or in combinations of two or more.

According to the present invention, a protein can be subjected to crosslinking treatment during and/or after nanoparticle formation. For the crosslinking treatment, an enzyme can be used. Any enzyme may be used without particular limitation as long as it has been known to have an action of causing protein crosslinking. Among such enzymes, transglutaminase is preferable.

Transglutaminase may be derived from a mammal or a microorganism. A recombinant transglutaminase can be used. Specific examples thereof include the Activa series by Ajinomoto Co., Inc., commercially available mammalian-derived transglutaminase serving as a reagent, such as guinea pig liver-derived transglutaminase, goat-derived transglutaminase, rabbit-derived transglutaminase, or human-derived recombinant transglutaminase produced by, for example, Oriental Yeast Co., Ltd., Upstate USA Inc., and Biodesign International.

The amount of an enzyme used for the crosslinking treatment in the present invention can be adequately determined depending upon protein type. In general, an enzyme can be added in a weight that is 0.1% to 100% and preferably approximately 1% to 50% of the protein weight.

The duration for an enzymatic crosslinking reaction can be adequately determined depending upon protein type and nanoparticle size. However, in general, the reaction can be carried out for 1 to 72 hours, and preferably 2 to 24 hours.

The temperature for an enzymatic crosslinking reaction can be adequately determined depending upon protein type and nanoparticle size. In general, the reaction can be carried out at 0° C. to 80° C. and preferably at 25° C. to 60° C.

Enzymes used in the present invention may be used alone or in combinations of two or more.

Nanoparticles of the present invention can be prepared in accordance with Patent Document: JP Patent Publication (Kokai) No. 6-79168 A (1994); or C. Coester, Journal Microcapsulation, 2000, vol. 17, pp. 187-193, provided that an enzyme is preferably used instead of glutaraldehyde for a crosslinking method.

In addition, according to the present invention, the enzymatic crosslinking treatment is preferably carried out in an organic solvent. The organic solvent used herein is preferably an aqueous organic solvent such as ethanol, isopropanol, acetone, or THF.

Further, according to the present invention, it is preferable to remove an organic solvent by distillation subsequent to a crosslinking treatment, followed by water dispersion. It is also possible to add water prior to or subsequent to removal of an organic solvent by distillation.

It is also possible to add at least one component selected from the group consisting of lipids (e.g., phospholipid), anionic polysaccharides, cationic polysaccharides, anionic proteins, cationic proteins, and cyclodextrin to the skin anti-aging agent for external use of the present invention. The amounts of lipid (e.g. phospholipid), anionic polysaccharide, cationic polysaccharide, anionic protein, cationic protein, and cyclodextrin to be added are not particularly limited. However, they can be added usually in a weight that is 0.1% to 100% of the protein weight. In the case of the skin anti-aging agent for external use of the present invention, it is possible to adjust the release rate by changing the ratio of the above components to the protein.

Specific examples of phospholipids that can be used in the present invention include, but are not limited to, the following compounds: phosphatidylcholine (lecithin), phosphatidylethanolamine, phosphatidylserine, phosphatidylinositol, phosphatidylglycerol, diphosphatidylglycerol, and sphingomyelin.

Anionic polysaccharides that can be used in the present invention are polysaccharides having an acidic polar group such as a carboxyl group, a sulfate group, or a phosphate group. Specific examples thereof include, but are not limited to, the following compounds: chondroitin sulfate, dextran sulfate, carboxymethyl cellulose, carboxymethyl dextran, alginic acid, pectin, carrageenan, fucoidan, agaropectin, porphyran, karaya gum, gellan gum, xanthan gum, and hyaluronic acids.

Cationic polysaccharides that can be used in the present invention are polysaccharides having a basic polar group such as an amino group. Examples thereof include, but are not limited to, the following compounds: polysaccharides such as chitin or chitosan, which comprise, as a monosaccharide unit, glucosamine or galactosamine.

Anionic proteins that can be used in the present invention are proteins and lipoproteins having a more basic isoelectric point than the physiological pH. Specific examples thereof include, but are not limited to, the following compounds: polyglutamic acid, polyaspartic acid, lysozyme, cytochrome C, ribonuclease, trypsinogen, chymotrypsinogen, and α-chymotrypsin.

Cationic proteins that can be used in the present invention are proteins and lipoproteins having a more acidic isoelectric point than the physiological pH. Specific examples thereof include, but are not limited to, the following compounds: polylysine, polyarginine, histone, protamine, and ovalbumin.

According to the present invention, it is possible to use casein nanoparticles prepared by the following steps (a) to (c) of:

(a) mixing casein with a basic aqueous medium at pH of 8 to less than 11;
(b) adding at least one active ingredient to the solution obtained in step (a); and
(c) injecting the solution obtained in step (b) into an aqueous medium at a pH of 3.5 to 7.5.

Further, according to the present invention, it is possible to use casein nanoparticles prepared by the following steps (a) to (c) of:

(a) mixing casein with a basic aqueous medium at a pH of 8 to less than 11;
(b) adding at least one active ingredient to the solution obtained in step (a); and
(c) lowering the pH of the solution obtained in step (b) to a pH value which is distinct from the isoelectric point by 1 unit or more.

According to the present invention, it is possible to prepare casein nanoparticles of desired sizes. Also, with the use of interaction between a hydrophobic active ingredient and a casein hydrophobic domain, it is possible for casein nanoparticles to contain the active ingredient. In addition, it was found that such particles remain stable in an aqueous solution.

Further, it was found that a particle mixture of casein and ionic polysaccharide or another ionic protein contains an ionic active ingredient.

The method for preparing casein nanoparticles of the present invention involves a method wherein casein is mixed with a basic aqueous medium solution and the solution is injected into another acidic aqueous medium, and a method wherein casein is mixed with a basic aqueous medium solution and the pH of the solution is lowered during stirring, for example.

The method wherein casein is mixed with a basic aqueous medium solution and the solution is injected into another acidic aqueous medium is preferably carried out using a syringe for convenience. However, there is no particular limitation as long as the injection rate, solubility, temperature, and stirring conditions are satisfied. Injection can be carried out usually at an injection rate of 1 mL/min to 100 mL/min. The temperature of the basic aqueous medium can be adequately determined. In general, the temperature is 0° C. to 80° C. and preferably 25° C. to 70° C. The temperature of an aqueous medium can be adequately determined. In general, the temperature can be 0° C. to 80° C. and preferably 25° C. to 60° C. The stirring rate can be adequately determined. However, in general, the stirring rate can be 100 rpm to 3000 rpm and preferably 200 rpm to 2000 rpm.

In the method wherein casein is mixed with a basic aqueous medium solution and the pH of the medium is lowered during stirring, it is preferable to add acid dropwise for convenience. However, there is no particular limitation as long as solubility, temperature, and stirring conditions are satisfied. The temperature of a basic aqueous medium can be adequately determined. However, in general, the temperature can be 0° C. to 80° C. and preferably 25° C. to 70° C. The stirring rate can be adequately determined. However, in general, the stirring rate can be 100 rpm to 3000 rpm and preferably 200 rpm to 2000 rpm.

The aqueous medium that can be used for the present invention is an aqueous solution or a buffer comprising an organic acid or base or an inorganic acid or base.

Specific examples thereof include, but are not limited to, aqueous solutions comprising: organic acids such as citric acid, ascorbic acid, gluconic acid, carboxylic acid, tartaric acid, succinic acid, acetic acid, phthalic acid, trifluoroacetic acid, morpholinoethanesulfonic acid, and 2-[4-(2-hydroxyethyl)-1-piperazinyl]ethanesulfonic acid; organic bases such as tris (hydroxymethyl), aminomethane, and ammonia; inorganic acids such as hydrochloric acid, perchloric acid, and carbonic acid; and inorganic bases such as sodium phosphate, potassium phosphate, calcium hydroxide, sodium hydroxide, potassium hydroxide, and magnesium hydroxide.

The concentration of an aqueous medium used in the present invention is preferably approximately 10 mM to 1 M, and more preferably approximately 20 mM to 200 mM.

The pH of a basic aqueous medium used in the present invention is preferably 8 or more, more preferably 8 to 12, and further preferably 9 to 11. When the pH is excessively high, there is concern regarding hydrolysis or risks in handling. Thus, the pH is preferably in the above range.

According to the present invention, the temperature at which casein is mixed with a basic aqueous medium at pH of 8 or more is preferably 0° C. to 80° C., more preferably 10° C. to 60° C., and further preferably 20° C. to 40° C.

The pH of an acidic aqueous medium used in the present invention is preferably 3.5 to 7.5 and more preferably 5 to 6.

The composition for transmucosal absorption of the present invention may further comprise an additive. Examples of an additive that can be used include, but are not limited to, soothing agents, preservatives, antioxidants, coloring agents, thickeners, aroma chemicals, and pH adjusters.

Specific examples of soothing agents that can be used in the present invention include, but are not limited to, the following compounds: benzyl alcohol, procaine hydrochloride, xylocalne hydrochloride, and chlorobutanol.

Specific examples of preservatives that can be used in the present invention include, but are not limited to, the following compounds: benzoic acid, sodium benzoate, paraben, ethylparaben, methylparaben, propylparaben, butylparaben, potassium sorbate, sodium sorbate, sorbic acid, sodium dehydroacetate, hydrogen peroxide, formic acid, ethyl formate, sodium hypochlorite, propionic acid, sodium propionate, calcium propionate, pectin degradation products, polylysine, phenol, isopropylmethyl phenol, orthophenylphenol, phenoxyethanol, resorcin, thymol, thiram, and tea tree oil.

Specific examples of antioxidants that can be used in the present invention include, but are not limited to, the following compounds: vitamin C and derivatives thereof, vitamin E, kinetin, polyphenol, SOD, phytic acid, BHT (di-n-butylhydroxytoluene), BHA (butylhydroxyanisole), propyl gallate, fullerene, and citric acid.

Specific examples of coloring agents that can be used in the present invention include, but are not limited to, the following compounds: krill pigment, orange dye, cacao dye, kaoline, carmines, ultramarine blue, cochineal dye, chrome oxide, iron oxide, titanium dioxide, tar dye, and chlorophyll.

Specific examples of thickeners that can be used in the present invention include, but are not limited to, the following compounds: quince seed, carrageenan, gum arabic, karaya gum, xanthan gum, gellan gum, tamarind gum, locust bean gum, gum traganth, pectin, starch, cyclodextrin, methylcellulose, ethylcellulose, carboxymethylcellulose, sodium alginate, polyvinyl alcohol, polyvinyl pyrrolidone, carboxyvinyl polymer, and sodium polyacrylate.

Specific examples of aroma chemicals that can be used in the present invention include, but are not limited to, the following compounds: musk, acacia oil, anise oil, ylang ylang oil, cinnamon oil, jasmine oil, sweet orange oil, spearmint oil, geranium oil, thyme oil, neroli oil, mentha oil, hinoki (Japanese cypress) oil, fennel oil, peppermint oil, bergamot oil, lime oil, lavender oil, lemon oil, lemongrass oil, rose oil, rosewood oil, anisaldehyde, geraniol, citral, civetone, muscone, limonene, and vanillin.

Specific examples of pH adjusters that can be used in the present invention include, but are not limited to, the following compounds: sodium citrate, sodium acetate, sodium hydroxide, potassium hydroxide, phosphoric acid, and succinic acid.

Specific examples of transmucosal administration may include administration via nasal mucosa, ocular mucosa, oral mucosa, pulmonary mucosa, vaginal mucosa, and digestive organ mucosa (for example, gastric mucosa, small intestinal mucosa, large intestinal mucosa, and rectal mucosa).

The dose of the composition for transmucosal absorption of the present invention can be adequately determined depending upon type and amount of active ingredient and upon user weight and condition, for example. The dose for single administration is generally approximately 1 μg to 50 mg/cm² and preferably 2.5 μg to 10 mg/cm².

The present invention is hereafter described in greater detail with reference to the following examples, although the technical scope of the present invention is not limited thereto.

### EXAMPLES

### Example 1

Acid-treated gelatin (10 mg), chondroitin sulfate-C (1 mg), transglutaminase preparation (5 mg; Activa TG-S, Ajinomoto Co., Inc.), adriamycin (0.4 mg; doxorubicin hydrochloride, Wako Pure Chemical Industries, Ltd.) and deionized water (1 ml) were mixed together. The resultant solution (1 ml) was injected into ethanol (10 mL) with the use of a microsyringe at an external temperature of 40° C. during stirring at 800 rpm. The resultant dispersion liquid was allowed to stand at an external temperature of 55° C. for 5 hours, so that crosslinked gelatin nanoparticles were obtained. The average particle size of the above particles was measured with a light scattering photometer (DLS-7000; Otsuka Electronics Co., Ltd.) and found to be 70 nm.

The nanoparticle dispersion liquid was centrifuged, and the supernatant ethanol was discarded. A physiological saline was added thereto, and the particles were dispersed again in such a way that the concentration of adriamycin is 200 μg/ml. The amount of adriamycin was calculated from the absorption spectra (Abs. 480 nm). The average particle size after redispersion was measured with a light scattering photometer (DLS-7000; Otsuka Electronics Co., Ltd.) and found to be 174 nm. Since the measurement of particle size by light scattering photometer was possible even in water solvent, it was demonstrated that enzymatic crosslinking reaction proceeded in the particles and crosslinked gelatin nanoparticles which is insoluble in water were prepared.

### Example 2

Casein Na (100 mg; Wako Pure Chemical Industries, Ltd.) was mixed with 50 mM phosphate buffer (pH 10, 10 mL). Tocopherol acetate (4.2 mg: Wako Pure Chemical Industries, Ltd.) was dissolved in ethanol (0.06 mL). The two different solutions were mixed together, and hydrochloric acid was added thereto to adjust the pH to pH7.5, so that casein nanoparticles were obtained.

The average particle size of the above particles was measured with a “Nantrac” light scattering photometer (NIKKISO Co., Ltd.) and found to be 36 nm.

### Example 3

Milk-derived casein (10 mg; Wako Pure Chemical Industries, Ltd.) was mixed with 50 mM phosphate buffer (pH 9, 1 mL). Tocopherol acetate (1.7 mg) was dissolved in ethanol (0.25 mL). The tocopherol acetate solution was added dropwise with stirring to the casein solution. The resultant solution (1 ml) was injected into 200 mM phosphate buffer (10 mL) with the use of a microsyringe at an external temperature of 40° C. during stirring at 800 rpm. Thus, aqueous dispersion of casein nanoparticles containing tocopherol acetate were obtained. The average particle size of the above particles was measured with a “Microtrac” light scattering photometer (NIKKISO Co., Ltd.) and found to be 124 nm.

### Example 4

Albumin (20 mg), chondroitin sulfate-C (2 mg), transglutaminase preparation (10 mg; Activa TG-S, Ajinomoto Co., Inc.), adriamycin (0.4 mg) and deionized water (1.79 ml) were mixed together. The resultant solution (1 ml) was injected into ethanol (10 mL) with the use of a microsyringe at an external temperature of 40° C. during stirring at 800 rpm. The resultant dispersion liquid was allowed to stand at an external temperature of 55° C. for 5 hours, so that crosslinked albumin nanoparticles were obtained. The average particle size of the above particles was measured with a light scattering photometer (DLS-7000; Otsuka Electronics Co., Ltd.) and found to be 30 nm.

### Example 5

Albumin (20 mg) was dissolved in 5 ml of 0.5M Tris-HCl buffer (pH8.5) containing 7M guanidine hydrochloride and 10 mM EDTA, and 20-mg of dithiothreitol was added thereto and mixed. Reduction was carried out at room temperature for 2 hours. The mixture was purified by gel filtration. To the resultant albumin solution was mixed chondroitin sulfate-C (2 mg) and adriamycin (0.4 mg). The resultant solution (1 ml) was injected into ethanol (10 mL) with the use of a microsyringe at an external temperature of 40° C. during stirring at 800 rpm. The resultant dispersion liquid was stirred in air at 40° C. for 3 hours, so that crosslinked albumin nanoparticles were obtained.

The average particle size of the above particles was measured with a light scattering photometer (DLS-7000; Otsuka Electronics Co., Ltd.) and found to be 200 nm.

### (Evaluation Method of Crosslinking Degree)

After purification by gel filtration, albumin concentration was measured by using Protein Assay Dye Reagent Concentrate (Bio Rad), and theoretical amount of SH was calculated. A calibration curve was prepared using glutathione as standard by using SH group color reagent DTNB (DOJINDO LABORATORIES), and the SH group in the albumin nanoparticles immediately after dispersion in ethanol was quantified. As a result, the amount of the SH group was almost consistent with the aforementioned theoretical amount which was calculated from the albumin amount. Thus, it was found that disulfide bond was reduced quantitatively. After stirring in air for 3 hours, the SH group in the albumin nanoparticles was quantified again. The amount of the SH group after stirring was compared with that before stirring. It was confirmed that 70% or more of disulfide bond was generated by air oxidation.

### Example 6

Acid-treated gelatin (10 mg) and transglutaminase preparation (5 mg; Activa TG-S, Ajinomoto Co., Inc.) were dissolved in water (1 ml). The resultant gelatin solution (1 ml) was injected into ethanol (10 mL) wherein glycyrrhetic acid (1.7 mg) was dissolved with the use of a microsyringe, so that gelatin nanoparticles were obtained. The resultant nanoparticles were allowed to stand at an external temperature of 55° C. for 5 hours, so that gelatin nanoparticles were crosslinked.

The average particle size of the above particles was measured with a “Microtrac” light scattering photometer (NIKKISO Co., Ltd.) and found to be 80 nm.

Water (5 ml) was added to the resultant gelatin nanoparticles dispersion liquid, and ethanol was removed by a rotary evaporator, so that an aqueous dispersion of gelatin nanoparticles containing glycyrrhetic acid therein was obtained.

The average particle size of the above particles was measured with a “Microtrac” light scattering photometer (NIKKISO Co., Ltd.) and found to be 201 nm.

### Example 7

Acid-treated gelatin (10 mg) and transglutaminase preparation (5 mg; Activa TG-S, Ajinomoto Co., Inc.) were dissolved in water (1 ml). The resultant gelatin solution (1 ml) was injected into ethanol (10 mL) wherein tocopherol. (1.7 mg) was dissolved with the use of a microsyringe, so that gelatin nanoparticles were obtained. The resultant nanoparticles were allowed to stand at an external temperature of 55° C. for 5 hours, so that gelatin nanoparticles were crosslinked.

The average particle size of the above particles was measured with a “Microtrac” light scattering photometer (NIKKISO Co., Ltd.) and found to be 95 nm.

Water (5 ml) was added to the resultant gelatin nanoparticles dispersion liquid, and ethanol was removed by a rotary evaporator, so that an aqueous dispersion of gelatin nanoparticles containing tocopherol therein was obtained.

The average particle size of the above particles was measured with a “Microtrac” light scattering photometer (NIKKISO Co., Ltd.) and found to be 240 nm.

### Example 8

Milk-derived casein (15 mg; Wako Pure Chemical Industries, Ltd.) was dissolved in phosphate buffer (pH 9, 1.5 mL). Astaxanthin (9 mg: Wako Pure Chemical Industries, Ltd.) was dissolved in ethanol (1 mL). The resultant two different solutions were mixed together. After ethanol was evaporated, the resulting liquid mixture (1 mL) was injected into phosphate buffer water (pH 5, 10 mL) with the use of microsyringe at an external temperature of 40° C. during stirring at 800 rpm. Thus, casein nanoparticles were obtained.

The average particle size of the above particles was measured with a “Microtrac” light scattering photometer (NIKKISO Co., Ltd.) and found to be 274 nm.

### Example 9

Milk-derived casein (15 mg; Wako Pure Chemical Industries, Ltd.) was dissolved in phosphate buffer (pH 9, 1.5 mL). Astaxanthin (9 mg: Wako Pure Chemical Industries, Ltd.) and tocopherol (2.75 mg) were dissolved in ethanol (1 mL). The resultant two different solutions were mixed together. After ethanol was evaporated, the resulting liquid mixture (1 mL) was injected into phosphate buffer water (pH 5, 10 mL) with the use of microsyringe at an external temperature of 40° C. during stirring at 800 rpm. Thus, casein nanoparticles were obtained.

The average particle size of the above particles was measured with a “Microtrac” light scattering photometer (NIKKISO Co., Ltd.) and found to be 293 nm.

### Example 10

Milk-derived casein (15 mg; Wako Pure Chemical Industries, Ltd.) was dissolved in phosphate buffer (pH 9, 1.5 mL). Astaxanthin (9 mg: Wako Pure Chemical Industries, Ltd.) and tocopherol (2.75 mg) were dissolved in ethanol (1 mL). The resultant two different solutions were mixed together. After ethanol was evaporated, the resulting liquid mixture (1 mL) was injected into phosphate buffer water (pH 5, 10 mL) with the use of microsyringe at an external temperature of 40° C. during stirring at 800 rpm. Thus, casein nanoparticles were obtained.

The average particle size of the above particles was measured with a “Microtrac” light scattering photometer (NIKKISO Co., Ltd.) and found to be 293 nm. Ascorbic acid (100 mg) was added to this dispersion liquid.

### Test Example 1

The dispersions of nanoparticles described in Examples 1 to 3 were preserved at room temperature for 1 month. Thereafter, the average particle size was measured using a Microtrac (NIKKISO Co., Ltd.).

As Comparative example 1, “NanoImpact” (Hosokawa Micron Corporation) which is nanoparticle dispersion of synthetic polymer (PLGA) was prepared.

Table 1 shows measurement results obtained in Test example 1.

TABLE 1

| | Comparative | Ex- | Ex- | Ex- |
|---|---|---|---|---|
| | example 1 | ample 1 | ample 2 | ample 3 |
| When | 600 nm | 70 nm | 36 nm | 124 nm |
| prepared | | | | |
| 1 month | N.D. | 71 nm | 42 nm | 148 nm |
| later | | | | |

N.D.: Not detectable

From the aforementioned results, it can be found that the particles of the composition for transmucosal absorption of the present invention are highly stable.

### Test Example 2

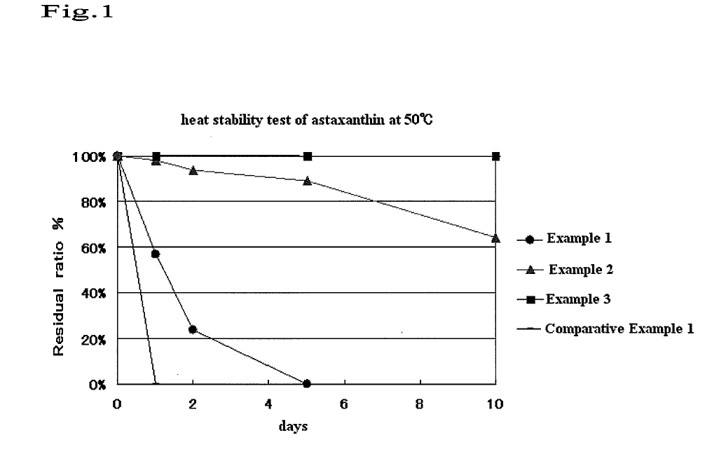

The casein nanoparticles prepared in Examples 8, 9 and 10 were left in thermostat bath at 50° C., and time course stability test was carried out for 10 days. In Comparative example, astaxanthin olive oil emulsion was used. The amount of astaxanthin was calculated from absorption spectra (Abs. 500 nm) (FIG. 1).

It can be found that casein nanoparticles show higher stability, as compared with the olive oil emulsion. Further, higher stability was shown by using an antioxidant (a stabilizing agent of anti-oxidizing compound) in combination, as compared with commercial available emulsion.

By incorporating astaxanthin having low stability into nanoparticles, the stability of astaxanthin can be increased. Further, its effect can be increased by addition of an additive.

Further, the safety is high since natural polymer is used. the transparency is high since the particle size is small.

1. A composition for transmucosal absorption which comprises protein nanoparticles containing an active ingredient and having an average particle size of 10 nm to 300 nm.

2. The composition for transmucosal absorption according to claim 1 wherein the protein nanoparticles contain an active ingredient in a weight that is 0.1% to 100% of the protein weight.

3. The composition for transmucosal absorption according to claim 1 wherein the active ingredient is at least one selected from the group consisting of functional food ingredients, and pharmaceutical ingredients.

4. The composition for transmucosal absorption according to claim 1 wherein the active ingredient is an ionic substance or a fat-soluble substance.

5. The composition for transmucosal absorption according to claim 1 wherein the protein is at least one selected from the group consisting of collagen, gelatin, acid-treated gelatin, albumin, ovalbumin, casein, transferrin, globulin, fibroin, fibrin, laminin, fibronectin, and vitronectin.

6. The composition for transmucosal absorption according to claim 1 wherein the protein is subjected to crosslinking treatment during and/or after nanoparticle formation.

7. The composition for transmucosal absorption according to claim 6 wherein crosslinking treatment is carried out by an enzyme.

8. The composition for transmucosal absorption according to claim 1 which comprises casein nanoparticles prepared by the following steps (a) to (c):
(a) mixing casein with a basic aqueous medium at pH of from 8 to less than 11;
(b) adding at least one active ingredient to the solution obtained in step (a); and
(c) injecting the solution obtained in step (b) into an aqueous medium at pH of 3.5 to 7.5:

9. The composition for transmucosal absorption according to claim 1 which comprises casein nanoparticles prepared by the following steps (a) to (c):
(a) mixing casein with a basic aqueous medium at pH of from 8 to less than 11;
(b) adding at least one active ingredient to the solution obtained in step (a); and
(c) lowering the pH of the solution obtained in step (b) to pH value which is different from the isoelectric point by 1 or more units.
